(19) United States
(12) Patent Application Publication
(10) Pub. No.: US 2017/0254810 A1
(43) Pub. Date: Sep. 7, 2017

(54) PROCESS FOR DETECTING PROSTATE CANCER BIOMARKERS

(71) Applicant: GOVERNMENT OF THE UNITED STATES, AS REPRESENTED BY THE SECRETARY OF COMMERCE, GAITHERSBURG, MD (US)

(72) Inventor: ERICA MCJIMPSEY, IV, MACOMB, IL (US)

(21) Appl. No.: 15/411,975

(22) Filed: Jan. 21, 2017

Related U.S. Application Data
(60) Provisional application No. 62/302,855, filed on Mar. 3, 2016.

(51) Int. Cl. G01N 33/574; G01N 33/68

(52) U.S. Cl. CPC G01N 33/57434; G01N 33/689

(57) ABSTRACT

A process for detecting prostate cancer biomarkers includes: subjecting seminal plasma fluid to western blot analysis; identifying a first prostate specific antigen (PSA) subform in the seminal plasma fluid, based on the western blot analysis; identifying a second PSA subform in the seminal plasma fluid, based on the western blot analysis; and quantifying a first amount of the first PSA subform and a second amount of the second PSA subform in the seminal plasma fluid to detect prostate cancer biomarkers in a subject from which the seminal plasma fluid was provided.

### CROSS REFERENCE TO RELATED APPLICATIONS

This application claims the benefit of U.S. Provisional Patent Application Ser. No. 62/302,855, filed Mar. 3, 2016, the disclosure of which is incorporated herein by reference in its entirety.

### STATEMENT REGARDING FEDERALLY SPONSORED RESEARCH

This invention was made with United States Government support from the National Institute of Standards and Technology. The Government has certain rights in the invention.

### BRIEF DESCRIPTION

Disclosed is a process for detecting prostate cancer biomarkers, the process comprising: subjecting seminal plasma fluid to western blot analysis; identifying a first prostate specific antigen (PSA) subform in the seminal plasma fluid, based on the western blot analysis; identifying a second PSA subform in the seminal plasma fluid, based on the western blot analysis; and quantifying a first amount of the first PSA subform and a second amount of the second PSA subform in the seminal plasma fluid to detect prostate cancer biomarkers in a subject from which the seminal plasma fluid was provided.

### BRIEF DESCRIPTION OF THE DRAWINGS

The following descriptions should not be considered limiting in any way. With reference to the accompanying drawings, like elements are numbered alike.

FIG. 1 shows a graphs of absorbance versus concentration;

FIG. 2 shows a graphs of absorbance versus concentration;

FIG. 3 shows an image of a gel resulting from subjecting a free prostate specific antigen (PSA) standards to sodium-dodecyl sulfate polyacrylamide gel electrophoresis (SDS-PAGE);

FIG. 4 shows an image of gels resulting from western blot (WB) testing; and

FIG. 5 shows an image of gels resulting from western blot (WB) testing.

### DETAILED DESCRIPTION

A detailed description of one or more embodiments is presented herein by way of exemplification and not limitation.

Prostate-specific antigen (PSA), the glycoprotein serum biomarker used for the detection of prostate cancer (PCa), has many different molecular forms. It has been discovered that a first internally cleaved molecular subform of PSA and a second internally cleaved molecular subform of PSA (collectively referred to as “prostate cancer biomarkers” and having respective molecular weights, e.g., of about 16 kDa and about 25 kDa) are downregulated in seminal plasma of men that have PCa. In an embodiment, a process for detecting PCa includes using western blot (WB) analysis; identifying these two PSA subforms from seminal plasma fluid; and quantifying these two PSA subforms from seminal plasma fluid to detect prostate cancer via the prostate cancer biomarkers.

Affinity interactions coupled with separation methods are important techniques for life science research and biotechnology. Affinity chromatography, Southern blotting, and gel-mobility shift assays are examples of widely used techniques that combine separation methodology with selective binding to improve information content and selectivity. Of the techniques that combine separations and affinity interactions, western blotting provides an assay of proteins in complex mixtures and provides a confirmatory test for clinical assays and regulatory tests.

In a western blot, proteins are separated by size using sodium dodecyl sulfate-polyacrylamide gel electrophoresis (SDS-PAGE) and then transferred to a membrane by electro-blotting (e.g., see Towbin et al, Proc. Natl. Acad. Sci. U.S.A. 76, 4350 (1979); Burnette, W. N., Anal. Biochem. 112, 195 (1981); and Gong et al, IEEE Sens. J. 8, 601 (2008)). The membrane is treated with blocking protein and then probed sequentially with primary and secondary antibody (conjugated with a label) to detect target protein. Western blotting provides selective protein detection based on size and antibody binding in a semi-quantitative assay or quantitative assay.

In an embodiment, WB analysis is used to test PSA subforms from normal and PCa seminal plasma samples. The samples can be stratified by age, race, and the like. Accordingly, embodiments involve developing age or race reference ranges for PSA subform concentrations.

In an embodiment, a test for prostate cancer (PCa) uses WB analysis to identify specific PSA molecular forms from seminal plasma. Here, WB analysis is used to identify specific proteins from a complex mixture of proteins in seminal plasma. During the WB analysis, proteins are separated, immobilized, and in subsequent steps detected by specific antibodies. Seminal plasma is a complex mixture of proteins that contains precursor forms of PSA, in addition to, free PSA that is composed of enzymatically active, intact PSA and enzymatically inactive, internally cleaved molecular subforms. PSA subforms are elevated in benign prostatic hyperplasia (BPH). We have discovered that a first PSA subform and a second PSA subform (which are two low abundant PSA subforms (˜16 kDa and ˜25 kDa)) present in non-cancerous seminal plasma sample is absent or downregulated in PCa seminal plasma samples (stage Tlc and 4). Moreover, the first PSA subform and the second PSA subform are elevated in the seminal plasma of men between the ages of 30 and 64, possibly due to BPH. In an embodiment, the first PSA subform and the second PSA subform are seminal plasma PCa disease markers, e.g., prostate cancer biomarkers. The process herein overcomes a problem where enzyme-linked immunosorbent assays (ELISA) that are used for the detection of PCa provide a false positive rate and lack specificity to differentiate BPH from PCa. In an embodiment, the WB test increases specificity of PCa detection, e.g., during early disease stages. Further, embodiments include absolute quantitation of proteins with high throughput at a sensitivity (picograms/milliliter) that is greater than or equal to ELISAs. In a particular embodiment, the process for detecting cancer includes using seminal plasma to quantify mass concentration in noncancerous specimens of target PSA subforms.

Embodiments overcome false positive rates of antibody detection of free and complexed PSA in equal molar ratios (equimolarity), as well as, measured PSA value discordances between immunoassay manufacturers.

We have discovered that the composition of the PSA molecular forms found in seminal plasma derived PSA immunoassay standards contribute to lack of PSA test reliability. Specifically, the WB results revealed that all of the four studied PSA standards contained different mass concentrations of intact and internally cleaved molecular forms. Increased mass concentrations of intact PSA yielded higher immunoassay absorbance values, even between lots from the same manufacturer. Increasing the reliability of the current PCa testing method includes standardization of the immunoassay (including, e.g., reagents, antibodies, calibrants, diluents, etc.). In an embodiment, a process for WB testing for PCa measures seminal plasma concentration of the first PSA subform and the second PSA subform. Advantageously, the process increases the specificity of PCa detection, substantially decreases or eliminates false positives, reduces overall cost of PCa diagnosis, and the like.

Beneficially and unexpectedly, the first PSA subform and the second PSA subform are used as biomarkers for detection of prostate cancer. In an embodiment, a process for detecting prostate cancer biomarkers includes: subjecting seminal plasma fluid to western blot analysis; identifying a first prostate specific antigen (PSA) subform in the seminal plasma fluid, based on the western blot analysis; identifying a second PSA subform in the seminal plasma fluid, based on the western blot analysis; and quantifying a first amount of the first PSA subform and a second amount of the second PSA subform in the seminal plasma fluid to detect prostate cancer biomarkers in a subject from which the seminal plasma fluid was provided.

In the process for detecting prostate cancer biomarkers, the first amount of the first PSA subform includes a seminal plasma concentration of the first PSA subform. The second amount of the second PSA subform includes a seminal plasma concentration of the second PSA subform. Further, the first PSA subform is formed in response to internally cleaving the PSA. Moreover, the second PSA subform is formed in response to cleaving the PSA. Additionally, the first PSA subform includes a molecular weight that is between 10 kiloDaltons (kDa) and 20 kDa. It is contemplated that the molecular weight of the first PSA subform is from 15 kDa to 17 kDa. In certain embodiments, the second PSA subform comprises a molecular weight that is between 20 kDa and 32 kDa and can be from 24 kDa to 26 kDa. The first PSA subform and the second PSA subform independently are enzymatically inactive and independently are an internally cleaved molecular subform of the PSA.

The process for detecting prostate cancer biomarkers also can include differentiating between prostate cancer biomarkers and benign prostatic hyperplasia. As a result of the process, a rate of a false positive for detecting prostate cancer biomarkers is less than or equal to 30%.

In the process for detecting prostate cancer biomarkers, subjecting seminal plasma fluid to western blot analysis includes separating seminal plasma proteins (that can include subforms thereof or intact PSA) on the basis of molecular weight by one-dimensional (1D) sodium dodecyl sulfate-polyacrylamide (SDS-PA) gel electrophoresis (1D SDS-PAGE). Proteins that are separated on the SDS-PA gel are transferred to a membrane, e.g., a polyvinylidene difluoride (PVDF) membrane. PSA is then complexed with a PSA primary antibody. Thereafter, the PSA primary antibody is complexed to a horse radish peroxidase-labeled secondary antibody that is used as a probe. A substrate is then added to the enzyme, and, once the substrate and enzyme are combined, light is produced as a byproduct. The light output is captured by digital chemiluminescence.

In the process for detecting prostate cancer biomarkers, identifying the first PSA subform in the seminal plasma fluid, based on the western blot analysis, includes inspecting (e.g., visually) for presence of a western blot band from 10 kDa to 20 kDa, wherein the band has a concentration that is less than a PSA calibrant band that has a molecular weight from 10 kDa to 20 kDa.

In the process for detecting prostate cancer biomarkers, identifying the second PSA subform in the seminal plasma fluid, based on the western blot analysis, includes inspecting (e.g., visually) for presence of a western blot band from 15 kDa to 32 kDa (more specifically 20 kDA to 32 kDA), wherein the band has a concentration that is less than a PSA calibrant band that has a molecular weight from 15 kDa to 32 kDa (more specifically 20 kDA to 32 kDA).

In the process for detecting prostate cancer biomarkers, quantifying the first amount of the first PSA subform and the second amount of the second PSA subform in the seminal plasma fluid includes comparing densitometrically chemiluminescence of western blot bands from 10 kDA to 20 kDa for the first PSA subform and from 15 to 32 kDa (more specifically from 20 kDA to 32 kDa) for the second PDS subform, wherein the comparison is performed between the seminal plasma sample and the PSA calibrant.

In the process for detecting prostate cancer biomarkers, the identity of the first PSA subform, the identity of the second PSA subform, and the first amount of the first PSA subform and the second amount of the second PSA subform are used to detect prostate cancer in a subject from which the seminal plasma fluid was provided by PSA subform identification of chemiluminescent western blot bands within the molecular mass ranges, 10 to 20 kDa and 15 to 32 kDa, that were determined by densitometric analysis to be lower in concentration (i.e., less dense) than the corresponding PSA subform calibrant. Similarly, prostate cancer staging is determined by densitometric comparative analysis of seminal plasma western blot bands and PSA calibrants from said relevant PSA subform molecular mass ranges, 10 to 20 kDa and 15 to 32 kDa.

In the process for detecting prostate cancer biomarkers, differentiating between prostate cancer biomarkers and benign prostatic hyperplasia consists of PSA subform identification by visual inspection and densitometric analysis of a chemiluminescent western blot band within the 10 to 20 kDa molecular mass range that is higher in concentration (i.e. more dense) than the corresponding PSA calibrant band.

Conventional PSA assays for detection of prostate cancer (PCa) lack specificity to differentiate PCa from benign prostatic hyperplasia and have high false positive rates. Calibrants for PSA used to create calibration curves in conventional assays are sometimes purified from seminal plasma and may contain molecular forms (intact PSA and cleaved subforms). It was believed that a composition of the PSA molecular forms found in conventional PSA standards contributed to lack of PSA test reliability. We investigated seminal plasma purified PSA standards from different commercial sources using western blot (WB) analysis and also did the same in multiple research grade PSA enzyme-linked immunosorbent assay (ELISA) (also referred to as enzyme immunoassay (EIA)). Results from WB analysis showed all of the PSA standards considered contained different mass concentrations of intact and cleaved molecular subforms. Increased mass concentrations of intact PSA yielded higher immunoassay absorbance values, even between lots from the same manufacturer. Standardization of seminal plasma derived PSA calibrant molecular form mass concentrations and purification methods alleviate differences in PCa testing measurements that use PSA values, such as the percentage (%) free PSA and prostate health index (PHI) by increasing the accuracy of the calibration curves.

For almost two decades, immunoassays that measure the serum level of the biomarker PSA have been used for the early detection and therapeutic monitoring of prostate cancer. PSA is an N-linked glycoprotein that includes a 237 amino acid residue of 28,400 Daltons (Da) (28.4 kiloDaltons (kDa)). Predominant immunoreactive forms of PSA, also referred to as isoforms, identified in serum include free (uncomplexed) PSA (fPSA) and PSA complexed to alpha 1-antichymotrypsin (PSA-ACT). In men who have PCa, PSA-ACT is elevated. Benign prostatic hyperplasia (BPH), a benign enlargement of the prostate, is associated with higher non-intact, free PSA serum levels. High false positive rates, problems with antibody detection of free and complexed PSA in equal molar ratios (equimolarity), as well as measured PSA value discordances between immunoassay manufacturers have been the subject of controversy as to whether use of conventional PSA tests is reliable. Identifying the source of inaccuracy in PSA measurements would provide increasing specificity of PCa testing. The calibration curve of a PSA immunoassay is involved in accurate measurement of an unknown mass concentration of serum PSA. Conventional calibration standards used in conventional clinical and research grade PSA immunoassays are either free PSA, a ratio of free and complexed PSA, or PSA of a recombinant form. Non-recombinant PSA protein standards are purified from seminal plasma. In addition to precursor forms of PSA, seminal plasma contains free PSA that is composed of enzymatically active, intact PSA and enzymatically inactive, nicked and clipped (internally cleaved) molecular subforms. These free PSA subforms are internally cleaved between residues 85-86, 145-146, and 182-183 in PSA. Seminal plasma derived PSA standards are purified from pooled (i.e., multiple donors) specimens, and PSA immunoassay calibrants based thereon can contain differences in concentrations of intact PSA or PSA molecular subforms. Lack of reliability in PSA immunoassays may be due to molecular differences in seminal plasma purified PSA calibrants. The process herein overcomes this shortcoming and provides a determination of differences in the composition of intact PSA and cleaved PSA subforms that create discordances in PSA ELISA mass concentration measurements. Moreover, PSA and PSA-ACT standards of a known mass concentration from different commercial sources were used as control standards (CS) and comparatively examined in several research grade total PSA (t-PSA) ELISAs. These immunoassays served as model clinical systems. Two of the assay calibrants, one of which was a recombinant PSA, were also comparatively investigated.

Total PSA ELISA Tests.

Three t-PSA ELISA kits (labelled as Calbiotech, #PS067T; R&D, #DKK300; Biocheck, #BC1029) were employed to evaluate PSA and PSA-ACT purified standards from different commercial sources. PSA calibrants from Calbiotech and R&D ELISAs were also examined. The Biocheck immunoassay was equimolar. The Calbiotech and R&D assays were non-equimolar. Immunoassay testing and kit storage was performed according to manufacturers' recommendations. Standards and calibrants were tested in triplicate. A microplate reader (commercially available as Safire 2 Microplate Reader from Tecan) collected raw absorbance values for all PSA standards and tested calibrants at a wavelength of 450 nm.

Commercial PSA and PSA-ACT CS.

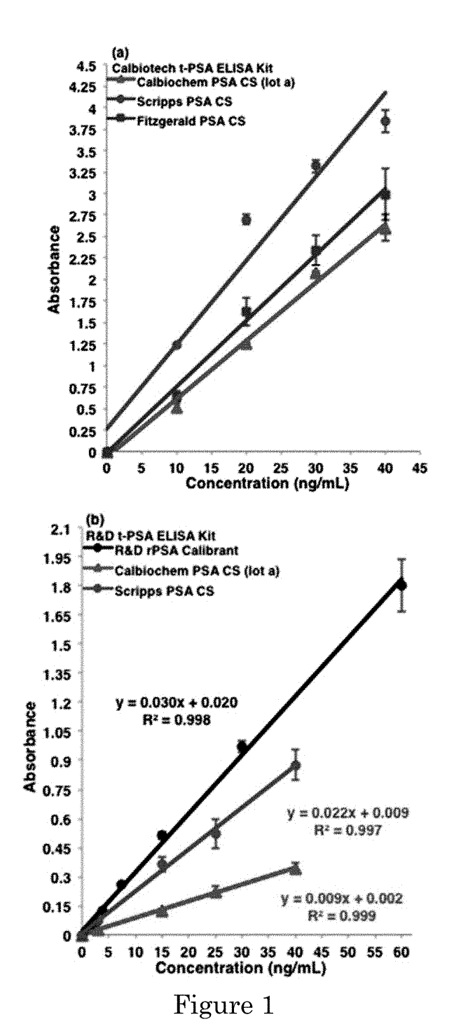

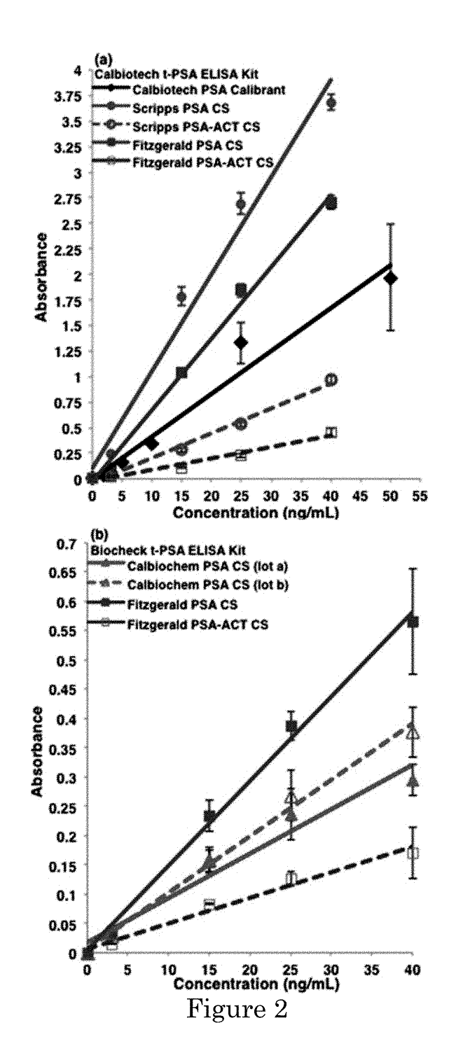

Four free PSA (Calbiochem, #539832 (lots D10010682 and D00116361); Scripps, #P0725; and Fitzgerald, #30R-AP019) and two PSA-ACT (Scripps, #P0625 and Fitzgerald, #30-AP13) CS were studied. The standards tested in the Calbiotech t-PSA ELISA kit (Calbiochem PSA (lot a), Scripps PSA, Scripps PSA-ACT, Fitzgerald PSA, and Fitzgerald PSA-ACT) were serially diluted with an in-house prepared 50 mM ammonium bicarbonate (Sigma, #09830) solution containing 0.067% bovine serum albumin (Thermo Scientific, #23210) and a protease inhibitor cocktail (Thermo, #78430) diluted to manufacturer recommendations. The standards tested in the R&D assay (Calbiochem PSA (lot a) and Scripps PSA) and Biocheck assay (Calbiochem PSA (lots a and b), Fitzgerald PSA, and Fitzgerald PSA-ACT) were serially diluted with the kit manufacturer's diluents. Final dilution concentrations for the CS were either ((10, 20, 30, and 40) ng/mL) (panel A of FIG. 1) or ((3, 15, 25, and 40) ng/mL) (panel B of FIG. 1 and panels A and B of FIG. 2).

FIG. 1 1 shows total PSA ELISA comparisons of free PSA CS in which average absorbance values are plotted against the commercially reported mass concentrations for three free PSA CS (Calbiochem, Scripps, and Fitzgerald) in the (panel A) Calbiotech and (panel B) R&D t-PSA immunoassays. One standard deviation is shown for each plotted test point. In both immunoassays, all of the PSA standards had different absorbance values. Additionally, FIG. 2 shows graphs of absorbance versus concentration for Calbiotech and Biocheck t-PSA ELISAs of free and complexed PSA CS.

Lower absorbance values were observed with the PSA-ACT CS in comparison to the PSA CS in both the Calbiotech and Biocheck t-PSA immunoassays. One standard deviation is shown for each plotted test point. In both immunoassays, the measured absorbance values for all of the PSA and PSA-ACT standards were different.

Prostate Cancer Samples.

Two serum samples were purchased from the biorepository, BioServe Biotechnologies, Limited. Samples from PCa donors with pathologic T stages of 2c (sample 1, #112417) and 3b (sample 2, #115218) were tested in duplicate using the R&D immunoassay.

Gel Electrophoretic Methods.

One-Dimensional PSA Gel.

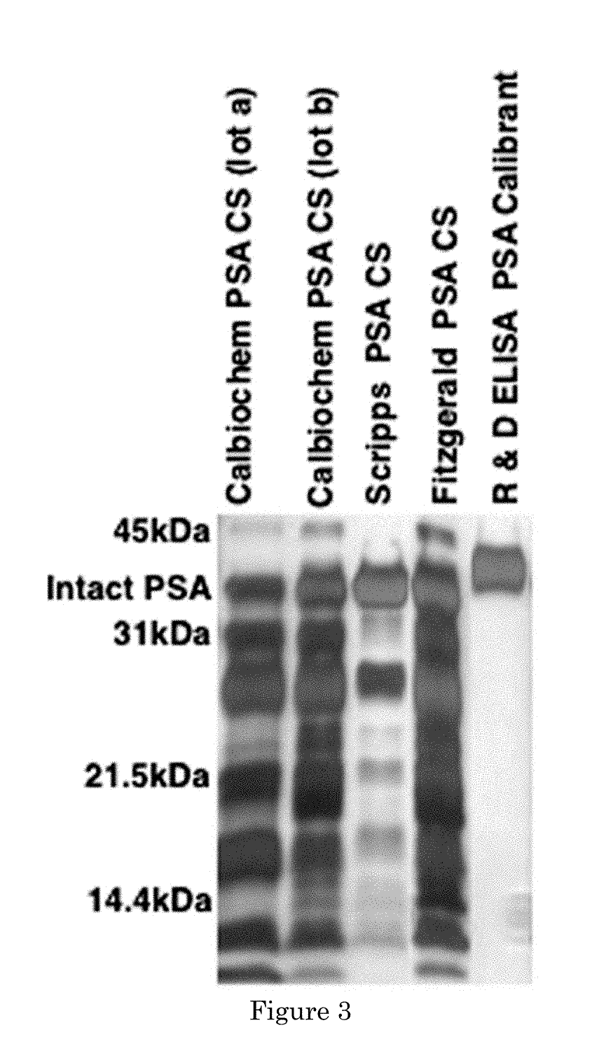

Two different lots of the Calbiochem PSA (D10010682 (lot a) and D00116361 (lot b)) CS as well as the Scripps and Fitzgerald PSA CS were studied by sodium dodecyl sulfate-polyacrylamide gel electrophoresis (SDS-PAGE). The R&D assay PSA calibrant (R&D, #1344-SE) was prepared according to manufacturer recommendations and was also examined (see results in FIG. 3). SDS-PAGE was performed in which samples (3 μg) were diluted in a 1:1 ratio with buffer (Bio-Rad Laboratories, #161-0737) under reducing conditions with 5% β-mercaptoethanol. PSA samples and 10 μL of protein molecular mass standards (Bio-Rad, #161-0137) were boiled for 5 minutes. After heating, samples were loaded on a 1.0 mm thick, 12.5% T/3.3% C polyacrylamide gel. Gels were run in a Mini Protean Tetra Electrophoresis Cell (Bio-Rad, #165-0827) and a voltage of 80 V for 15 min followed by 80 min at 120 V was applied. Proteins were silver stained and destained (Thermo, #24612) according to manufacturer recommendations.

Accordingly, FIG. 3 shows SDS-PAGE results for free PSA standards in which differences in the relative concentrations of PSA isoforms and subforms were observed by SDS-PAGE for the free PSA CS from Calbiochem (2 different lots), Scripps, and Fitzgerald, as well as, the R&D ELISA rPSA calibrant.

Western Blots of PSA and PSA-ACT.

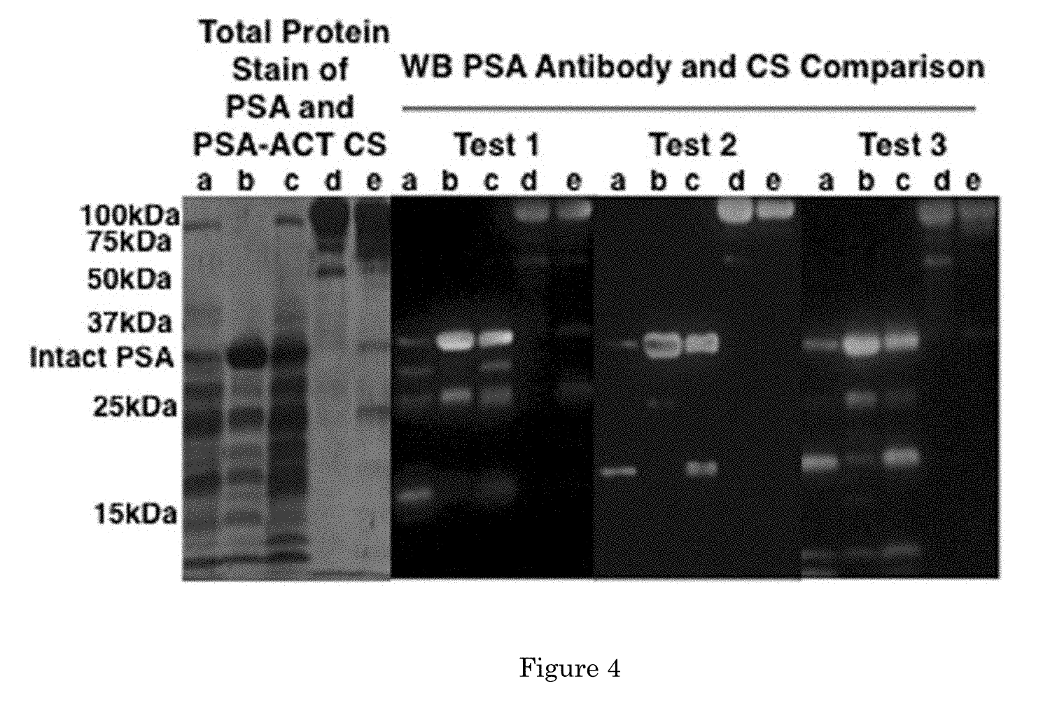

SDS-PAGE was performed on molecular mass standards (Bio-Rad, #161-0375), PSA CS (Calbiochem (lot a), Scripps, and Fitzgerald), and PSA-ACT CS (Scripps and Fitzgerald) as described above. Gel electrophoresis was run at a voltage of 80 V for 15 min followed by 70 min at 120 V. Gel protein bands were transferred onto polyvinylidene fluoride (PVDF) membranes (Bio-Rad, #162-0177) using a tank blotting system (Bio-Rad, #165-0827) in a 20% MeOH/buffer solution at a constant voltage of 60 for 2 hours. A western blot (WB) detection system (GE Healthcare, RPN2135) was used according to manufacturer recommendations. PVDF membranes were blocked with a 2% blocking agent (Bio-Rad, #170-6404) for 45 min. WB primary antibodies were diluted at a ratio of 1:5000 (Abcam, ab28563), 1:900 (Santa Cruz (SC) Biotechnology Incorporated, CHYH1, sc-69663), and 1:1600 (SC, C-19, sc-7638) for antibody tests 1, 2, and 3, respectively (see results shown in FIG. 4). The corresponding secondary antibodies were diluted 1:100000 (SC, sc-2004), 1:2900 (SC, sc-2005), and 1:18500 (SC, sc-2768) for antibody tests 1, 2, and 3, respectively. WB PVDF membrane imaging was performed on an Alpha Innotech Fluorchem SP Imager. A total protein stain (Bio-Rad, #170-6527) was applied overnight to membranes from the WB antibody tests to confirm complete transfer of protein.

FIG. 4 shows results for WB analysis of free and complexed PSA CS in which a total protein membrane stain of the PSA and PSA-ACT CS after WB test 1 is shown. WB studies using three different antibodies against t-PSA (Test 1, 2, and 3) for PSA CS from (panel A) Calbiochem (lot a), (panel B) Scripps, and (panel C) Fitzgerald, as well as, PSA-ACT CS from (panel D) Scripps and (panel E) Fitzgerald are shown.

As shown in FIG. 1, the CS have different absorbance signals when tested in the Calbiotech and R&D immunoassays. The analytical response in absorbance for each of the three CS analyzed in the Calbiotech assay varied. The strongest absorbance signal was observed with the Scripps PSA while the Calbiochem PSA (lot a) exhibited the weakest. As was the case with the Calbiotech assay (panel A of FIG. 1), the Scripps PSA had higher measured absorbance values relative to the Calbiochem PSA in the R&D assay (panel B of FIG. 1). The recombinant PSA (rPSA) calibrant had the highest absorbance signals. Two PCa serum samples were also tested in the R&D assay. The absorbance values for the t-PSA blood levels are shown in the Table below that lists PCa serum t-PSA measurement comparisons.

| | | | | TABLE | | |
|---|---|---|---|---|---|---|
| | | | | t-PSA (ng/mL) Measurement | | |
| | | | | Comparison Using Linear | | |
| | | | | Regression Equations from R&D | | |
| | | | | Immunoassay PSA Calibrant and CS | | |
| PCa | Pathologic | Measured | | R&D | | Scripps |
| Serum | Tumor Stage of | Absorbance | Coefficient | rPSA | Calbiochem | PSA |
| Samples | PCa Serum Donor | of t-PSA | of Variation | Calibrant | PSA CS | CS |
| 1 | 2c | 0.8 | 9.6 | 4 | 17.4 | 6.5 |
| 2 | 3b | 0.6 | 1.7 | 38.9 | 133.5 | 54 |

The t-PSA serum levels of two PCa samples examined in the R&D ELISA were computed using the linear regression equations from the R&D ELISA rPSA calibrant and R&D tested PSA CS.

The t-PSA concentrations were computed using the linear regression equations of the Calbiochem and Scripps PSA standards, as well as, the rPSA immunoassay calibrant. The concentrations ranged between 4.0 ng/mL and 17.4 ng/mL for PCa serum sample 1 and 38.9 ng/mL and 133.5 ng/mL for sample 2. Complexed PSA was also studied in the Calbiotech (panel A of FIG. 2) and Biocheck (panel A of FIG. 2) assays. In the Calbiotech assay, both the Scripps and Fitzgerald PSA standards absorbed more strongly than the Scripps and Fitzgerald PSA-ACT standards (panel A of FIG. 2). A comparison of the Calbiotech and Biocheck immunoassays yielded similar results for the Fitzgerald PSA and PSA-ACT standards. In the Biocheck ELISA (panel B of FIG. 2), an equimolar assay, the absorbance signal of the Fitzgerald PSA-ACT standard was attenuated in comparison to the Fitzgerald PSA CS. Lot to lot variations in the Calbiochem PSA were also examined in the Biocheck assay (panel B of FIG. 2). The Calbiochem PSA CS (lot b) had slightly higher absorbance values than the Calbiochem PSA CS (lot a).

To determine the source of the absorbance variances between the PSA standards, a one-dimensional (1D) SDS-PAGE was performed on both of the Calbiochem PSA CS lots as well as the Scripps and Fitzgerald standards (FIG. 3). The R&D r-PSA calibrant was also examined. The intact PSA bands, observed at approximately 34 kDa, were higher than the molecular mass of free PSA; glycosylated proteins are known to migrate differently than non-glycosylated proteins on SDS-PAGE. The relative subform abundances of the two Calbiochem PSA CS lots varied and more intact PSA was observed in lot b than lot a. Similarly, the Scripps and Fitzgerald PSA standards had varying compositions of PSA molecular forms. The R&D r-PSA calibrant contained only the intact molecular form of PSA. PSA molecular form compositions were also examined by WB (FIG. 4). WB tests were performed on the three PSA and two PSA-ACT CS studied by ELISA using three different antibodies against both PSA and PSA-ACT (WB tests 1, 2, and 3). One representative total protein membrane stain, confirming the complete transfer of PSA standard, is shown for WB test 1 (FIG. 4). Antibody dependent differences in the presence and/or absence of PSA subforms were observed for each of the standards. Intact PSA was observed at approximately 34 kDa (panels A, B, and C of FIG. 4). Through the collective use of three PSA antibodies, up to 8 unique bands ranging in molecular mass between approximately 10 and 31 kDa were confirmed as subforms for each free PSA standard. Intact PSA-ACT bands were observed at approximately 100 kDa (panels D and E of FIG. 4). PSA, as well as, PSA-ACT subforms were also present in the PSA-ACT standards (panels D and E of FIG. 4). The relative amount of intact PSA present was determined through visual inspection and based on WB protein band size (FIG. 4). Both the intact PSA and cleaved subform concentrations varied by commercial source. The relative concentrations of intact free PSA observed between manufacturers ([Calbiochem PSA (lot a)]<[Fitzgerald PSA]<[Scripps PSA]) were consistent for each of the three PSA antibodies studied (FIG. 4). Similarly, the relative concentration of intact Scripps PSA-ACT was determined to be higher than that of the Fitzgerald PSA-ACT CS.

The PSA standard absorbance trends observed between assays were determined to be correlated with the concentration of intact PSA and PSA-ACT present in the standards, even between lots, in the case of the Calbiochem PSA standards (panel A of FIG. 2). In the Calbiotech assay (panel A of FIG. 1), the intensity of the free PSA standard absorbance signals from weakest to strongest was Calbiochem PSA (lot a)<Fitzgerald PSA<Scripps PSA. The WB studies (FIG. 4) revealed that the relative concentrations from lowest to highest of intact PSA for the PSA standards was [Calbiochem PSA (lot a)]<[Fitzgerald PSA]<[Scripps PSA]). When measured in the R&D assay (panel B of FIG. 1), the PSA absorbance trends were Calbiochem PSA (lot a)<Scripps PSA<R&D rPSA. The R&D r-PSA calibrant was determined by SDS-PAGE to be composed solely of intact PSA and did not contain any internally cleaved subforms whereas the Scripps and Calbiochem PSA CS were composed of many (FIG. 3). In the Biocheck assay (panel B of FIG. 2), the Fitzgerald PSA standard had higher absorbance signals in relation to the Calbiochem PSA standards for both lots a and b. The Calbiochem PSA lot b standard, however, had higher immunoassay absorbance values (panel B of FIG. 2) and a higher relative concentration of intact PSA in comparison to lot a (FIG. 3). Lot to lot differences in the concentration of intact PSA between PSA standards from the same company can therefore create immunoassay absorbance differences. The Fitzgerald and Scripps PSA and PSA-ACT standards had similar absorbance and intact molecular form concentration trends. In the Calbiotech assay (panel A of FIG. 2), the Scripps PSA-ACT standard had a higher absorbance than the Fitzgerald PSA-ACT CS. By WB, the concentration of the intact Scripps PSA-ACT was observed to be higher than that of the Fitzgerald PSA-ACT standard. In short, increased concentrations of intact PSA and PSA-ACT in the standards resulted in increases in immunoassay absorbance values. Lower concentrations of intact PSA and PSA-ACT in standards resulted in lower immunoassay absorbance values. These findings suggest that the PSA immunoassay antibodies are preferentially binding to the intact PSA and intact PSA-ACT molecular forms.

Although the WHO First International Standards for PSA (90% PSA-ACT: 10% free PSA (also known as 90:10) and free PSA) were established as calibrants to standardize PSA diagnostic immunoassays that were nonequimolar in response to PSA and PSA-ACT, interassay disparities in free and total PSA measurements have continued. In the Biocheck t-PSA ELISA results presented herein, the absorbance signal of the Fitzgerald PSA-ACT standard was attenuated in comparison to the Fitzgerald PSA standard, despite assay equimolarity. In addition to immunoassay nonequimolarity, some possible other causes for these absorbance differences include dissociation of PSA-ACT or inaccurate assignment of PSA-ACT mass concentration values. A large majority of the research grade and diagnostic PSA immunoassays are likely not equimolar in response to the intact molecular forms and cleaved subforms of PSA and PSA-ACT.

The WHO PSA standards may yield 20-25% lower t-PSA and fPSA test results when calibrated against the Hybritech standards in the Access Immunoassay System. Different ratios of PSA and PSA-ACT, though equal in PSA concentration, may contain varying amounts of intact PSA and PSA-ACT that can produce equimolar disparities. These same diagnostic immunoassays, including but not limited to, Abbott Architect, Bayer Immuno 1, Beckman Hybritech, Perkin Elmer DELFIA, Roche E170, and Ortho Clinical Diagnostics are FDA approved and in use within the United States.

The observed molecular form composition differences between seminal plasma derived PSA standards, specifically the concentration of intact PSA, may be a result from PSA standards being purified from pooled specimens that vary in accordance with the individual donor's intact PSA and cleaved subform makeup. The use of various purification processes of PSA by different manufacturers may alter the composition of the PSA standard molecular forms present.

The PCa serum sample PSA value findings (see Table) provide that molecular form differences between PSA standards can create intraassay discordances in t-PSA measurements that can provide an unreliable PSA serum level report from a clinical laboratory. For PCa serum sample 1, the computed t-PSA concentrations ranged between 4.0 ng/mL and 17.4 ng/mL, depending on the calibration curve used. Deviations from accuracy of the t-PSA (free PSA+PSA-ACT) serum concentration can result in inaccurate % fPSA (free PSA/free PSA+PSA-ACT) reporting. It is contemplated that, for % fPSA, the measurement used to differentiate BPH from PCa is a calculation of the PSA concentrations from two different PSA immunoassays that rely on two distinct calibration curves and PSA calibrants to determine the unknown free and complexed PSA serum levels. The cumulative effects of the experimental differences found between the free and t-PSA immunoassays, due to variations in standards, challenges the utility of % fPSA numerical ratio values.

Use of any nonequimolar PSA immunoassay that contains a seminal plasma derived PSA calibrant could result in skewed PSA serum level reporting leading to either over diagnosis or under diagnosis of PCa. Derivatives of the PSA test have been developed to increase the specificity of PCa detection, including but not limited to, % fPSA, PSA velocity (PSAV), and the prostate health index (PHI). The PSAV, a measure of the rate of change in PSA levels, has been shown to only minimally, in comparison to % fPSA, increase the predictive accuracy of early PCa detection (AUC of 0.626 vs 0.609). The PHI, a newly FDA approved test for the early detection of PCa in the 2.0 to 10.0 ng/mL PSA range, has shown more promise. This test utilizes a score based on the mathematical formula, ([−2]proPSA/free PSA)×√PSA), which combines results from three individual PSA measurements (t-PSA, % fPSA, and a truncated precursor form of fPSA containing a leader sequence of 2 amino acids known as [−2]proPSA). Although at a sensitivity of 90%, specificity of PHI may be 31%, in comparison to 11% and 10% for % fPSA and t-PSA, respectively, and a difference in the reduction of false positives may exist. With regard to improvement in screening of PCa using the PHI, the [−2]proPSA measurement is performed through use of a recombinant [−2]proPSA calibrant and an anti [−2]proPSA antibody that exhibits low cross reactivity to free PSA (<0.2%) and other truncated precursor forms of PSA (<2%). This high specificity of the anti [−2]proPSA antibody to [−2]proPSA removes potential downstream problems of preferential binding to the antibody from other serum PSA molecular forms. Use of an accurate [−2]proPSA calibration curve, as well as, the accurate measurement of serum [−2]proPSA provides increased specificity of the PHI test in the discrimination of PCa. Conventional PCa screenings use longitudinal monitoring of PSA, such as PSAV and PSA doubling time, and are of diagnostic benefit if, with repeated measurements, the clinical laboratory, PSA assay, and molecular form concentrations of the PSA and PSA-ACT calibrant remain exactly the same.

Until PSA immunoassays are equimolar in response to intact PSA and PSA-ACT, as well as, the precursor forms and cleaved molecular subforms of PSA and PSA-ACT, standardization of molecular form mass concentrations to known and absolute values is needed in seminal plasma derived PSA calibrants to help reduce PSA level reporting errors. Upon determination of the molecular form ratios and mass concentrations present in serum fPSA, as well as, serum fPSA complexed to ACT during early stage PCa, better standardization of PSA immunoassay calibrants could be achieved. Preparation of a fPSA calibrant involves a multistep process and include seminal plasma purification, isolation, and quantification of the previously determined fPSA molecular forms present in PCa followed by recombination of the molecular forms in defined mass concentrations. The same general protocol for preparation of a PSA-ACT calibrant can be used with the exception that complexation of the fPSA to ACT would occur prior to combining the PSA molecular forms. For the t-PSA immunoassay, use of a calibrant containing the already adopted isoform ratio of 90% PSA-ACT and 10% fPSA with the previously identified molecular form ratios and mass concentrations for each isoform would best be employed.

Accordingly, the intact PSA and intact PSA-ACT molecular forms of the PSA standards examined were bound preferentially in several t-PSA immunoassays, regardless of assay equimolarity. Diagnostic use of PSA immunoassays that exhibit a nonequimolar response to various molecular forms of PSA and PSA-ACT result in unreliable PSA serum level reporting if the immunoassay calibrants were not standardized to known and absolute molecular form ratios and mass concentrations. Molecular form ratio and mass concentration standardization of PSA immunoassay calibrants is contemplated and assist in increasing the specificity of PCa testing. To achieve better uniformity between laboratories, standardization of a seminal plasma PSA purification protocol also is an unexpected benefit.

The process for detecting prostate cancer biomarkers has numerous advantageous and beneficial properties. The process has a low false positive rate for detection of prostate cancer, differentiates between prostate cancer and BPH, and provides (in the case of prostate cancer) determination of the stage of prostate cancer that the subject that provided the seminal plasma has.

The articles and processes herein are illustrated further by the following Example, which is non-limiting.

### Example

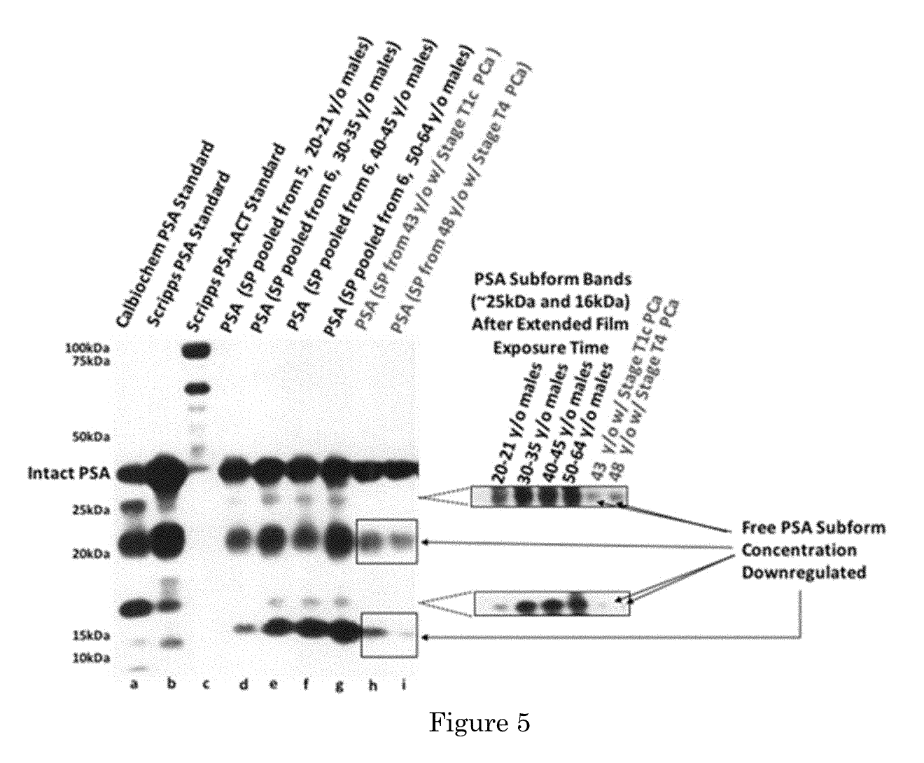

Prostate cancer biomarkers were detected in a seminal plasma sample. Here, WB analysis provided results from the seminal plasma samples as shown in FIG. 5. Four PSA molecular subforms (˜15 kD, ˜16 kD, ˜20 kD, and ˜25 kD) present in non-cancerous seminal plasma samples (panels d-g) are downregulated in PCa seminal plasma samples. Two representative PCa samples (T1c and T4) are shown (panels h and i, respectively). These PSA subforms served as seminal plasma PCa markers. The PSA subform concentration at ˜15 kDa increased with age and decreased with PCa disease. Increase in subform concentration with age was attributed to BPH. PSA subform at ˜15 kDa was a biomarker for BPH detection. Additionally, seminal plasma levels of PSA subforms at ˜15 kDa and ˜16 kDa were downregulated in accordance with PCa stage progression. Moreover, the relative concentration of the PSA subforms was lower with the stage T4 PCa sample in comparison to the stage T1c PCa sample. With each of the PSA subform markers identified (˜15 kD, ˜16 kD, ˜20 kD, and ˜25 kD), the T1c PCa sample protein concentration is downregulated in comparison to the noncancerous seminal plasma samples (ages 30-64).

While one or more embodiments have been shown and described, modifications and substitutions may be made thereto without departing from the spirit and scope of the invention. Accordingly, it is to be understood that the present invention has been described by way of illustrations and not limitation. Embodiments herein can be used independently or can be combined.

Reference throughout this specification to “one embodiment,” “particular embodiment,” “certain embodiment,” “an embodiment,” or the like means that a particular feature, structure, or characteristic described in connection with the embodiment is included in at least one embodiment. Thus, appearances of these phrases (e.g., “in one embodiment” or “in an embodiment”) throughout this specification are not necessarily all referring to the same embodiment, but may. Furthermore, particular features, structures, or characteristics may be combined in any suitable manner, as would be apparent to one of ordinary skill in the art from this disclosure, in one or more embodiments.

All ranges disclosed herein are inclusive of the endpoints, and the endpoints are independently combinable with each other. The ranges are continuous and thus contain every value and subset thereof in the range. Unless otherwise stated or contextually inapplicable, all percentages, when expressing a quantity, are weight percentages. The suffix “(s)” as used herein is intended to include both the singular and the plural of the term that it modifies, thereby including at least one of that term (e.g., the colorant(s) includes at least one colorants). “Optional” or “optionally” means that the subsequently described event or circumstance can or cannot occur, and that the description includes instances where the event occurs and instances where it does not. As used herein, “combination” is inclusive of blends, mixtures, alloys, reaction products, and the like.

As used herein, “a combination thereof” refers to a combination comprising at least one of the named constituents, components, compounds, or elements, optionally together with one or more of the same class of constituents, components, compounds, or elements.

All references are incorporated herein by reference.

The use of the terms “a” and “an” and “the” and similar referents in the context of describing the invention (especially in the context of the following claims) are to be construed to cover both the singular and the plural, unless otherwise indicated herein or clearly contradicted by context. “Or” means “and/or.” Further, the conjunction “or” is used to link objects of a list or alternatives and is not disjunctive; rather the elements can be used separately or can be combined together under appropriate circumstances. It should further be noted that the terms “first,” “second,” “primary,” “secondary,” and the like herein do not denote any order, quantity, or importance, but rather are used to distinguish one element from another. The modifier “about” used in connection with a quantity is inclusive of the stated value and has the meaning dictated by the context (e.g., it includes the degree of error associated with measurement of the particular quantity).

What is claimed is:

1. A process for detecting prostate cancer biomarkers, the process comprising:
subjecting seminal plasma fluid to western blot analysis;
identifying a first prostate specific antigen (PSA) subform in the seminal plasma fluid, based on the western blot analysis;
identifying a second PSA subform in the seminal plasma fluid, based on the western blot analysis; and
quantifying a first amount of the first PSA subform and a second amount of the second PSA subform in the seminal plasma fluid to detect prostate cancer biomarkers in a subject from which the seminal plasma fluid was provided.

2. The process for detecting prostate cancer biomarkers of claim 1, wherein the first amount of the first PSA subform comprises a seminal plasma concentration of the first PSA subform.

3. The process for detecting prostate cancer biomarkers of claim 2, wherein the second amount of the second PSA subform comprises a seminal plasma concentration of the second PSA subform.

4. The process for detecting prostate cancer biomarkers of claim 3, wherein the first PSA subform is formed in response to internally cleaving the PSA.

5. The process for detecting prostate cancer biomarkers of claim 4, wherein the second PSA subform is formed in response to cleaving the PSA.

6. The process for detecting prostate cancer biomarkers of claim 5, wherein the first PSA subform comprises a molecular weight that is between 10 kiloDaltons (kDa) and 20 kDa.

7. The process for detecting prostate cancer biomarkers of claim 6, wherein the molecular weight of the first PSA subform is from 15 kDa to 17 kDa.

8. The process for detecting prostate cancer biomarkers of claim 7, wherein the second PSA subform comprises a molecular weight that is between 20 kDa and 32 kDa.

9. The process for detecting prostate cancer biomarkers of claim 8, wherein the molecular weight of the second PSA subform is from 24 kDa to 26 kDa.

10. The process for detecting prostate cancer biomarkers of claim 7, wherein the first PSA subform and the second PSA subform independently are enzymatically inactive and independently are an internally cleaved molecular subform of the PSA.

11. The process for detecting prostate cancer biomarkers of claim 10, further comprising:
differentiating between prostate cancer biomarkers and benign prostatic hyperplasia.

12. The process for detecting prostate cancer biomarkers of claim 11, wherein a rate of a false positive for detecting prostate cancer biomarkers is less than or equal to 30%.
